(19) United States
(12) Patent Application Publication
(10) Pub. No.: US 2018/0050145 A1
(43) Pub. Date: Feb. 22, 2018

(54) APPARATUS FOR EXTRACORPOREAL BLOOD TREATMENT COMPRISING A MONITOR UNIT

(71) Applicant: B. BRAUN AVITUM AG, MELSUNGEN (DE)

(72) Inventors: OLIVER SCHÄFER, Neuenstein (DE); Christian Schleicher, Dipperz (DE)

(21) Appl. No.: 15/678,319

(22) Filed: Aug. 16, 2017

(30) Foreign Application Priority Data
Aug. 17, 2016 (DE) 10 2016 115 270.6

(51) Int. Cl. A61M 1/16; A61M 1/36; A61M 1/34

(52) U.S. Cl. CPC A61M 1/1621; A61M 2205/3331; A61M 1/34; A61M 1/3653

(57) ABSTRACT

An apparatus for extracorporeal blood treatment includes a body and a monitor unit fixed in the tilt direction which includes a display unit. The apparatus for extracorporeal blood treatment may be configured as a dialysis machine including a housing for accommodating operational components for the extracorporeal blood treatment. The monitor unit includes a display content representation influencing device.

### CROSS REFERENCE TO RELATED APPLICATIONS

This application claims priority to German application DE 10 2016 115 270.6 filed Aug. 17, 2016, the contents of such application being incorporated by reference herein.

### FIELD OF THE INVENTION

The invention relates to an apparatus for extracorporeal blood treatment such as a dialysis machine which includes a monitor unit in the form of a screen having a display unit, for example.

### BACKGROUND OF THE INVENTION

From the state of the art, fixed monitor units are known in medical apparatuses, for example machines for extracorporeal blood treatment, such as e.g. dialysis machines. Those fixed or at least non-tiltable monitors of the known type have been a drawback so far to the effect that in adverse conditions, for example with adverse incidence of light on a display unit of the fixed monitor or with an adverse position of the apparatus, a user interface is not or only hardly visible, and the read-out of information shown on the display unit and/or of a user interface is impaired and the information cannot be clearly identified and/or is difficult to identify.

Since it is not possible to simply rotate the monitor toward a more favorable incidence of light with a fixed monitor and usually the rotatability of the apparatus in situ is equally limited, rotation of the entire apparatus which might improve the read-out may result, in a further detrimental manner, in the fact that for example information contrary to data privacy regulations, e.g. confidential case data, will become accessible to persons for whom it is not meant.

In order to counteract this fact, known apparatuses, such as dialysis machines, therefore should be orientated separately so that they are not visible by non-authorized persons. Although in the event of interconnected machines, treatment parameters and/or case data, for example, may be presentable and visible with a network interface in a nurse room, for example. However, it has not been possible so far to see, directly on a machine, case data and/or parameters of a neighboring machine without revealing information in an undesired way or endangering the compliance with data privacy regulations.

### SUMMARY OF THE INVENTION

Consequently, an object underlying the invention is to overcome the afore-mentioned drawbacks and, in an apparatus for extracorporeal blood treatment such as a dialysis machine comprising a fixed monitor unit, to improve the read-out of display contents on a display unit thereof in a practically useful and user-friendly manner, while at the same time increasing data privacy.

This object is achieved, according to aspects of the invention, by an apparatus for extracorporeal blood treatment such as a dialysis machine comprising the features of the independent claim. Advantageous developments of the invention are the subject matter of the enclosed subclaims.

The general idea underlying the invention is to improve in a fixed and/or rotatable monitor, which is not tiltable/non-tiltable, with antireflection coating such as a specific film, a coating or the like, for example, the visibility of a user interface, i.e. on an apparatus for extracorporeal blood treatment, such as a dialysis machine and the like which includes a fixed and/or integrated monitor unit (monitor) being adapted to be non-orientable and including a display unit, to influence the read-out of information represented on the display unit by well-directed measures or an interacting combination of well-directed measures. The well-directed measures comprise a representation of information depending on the viewing angle via controlled liquid crystals, a reduction of reflection or antireflection coating and/or polarization of a display surface by applying or integrating e.g. layers or films to/in the display surface or arranging a layer or film in front of the display surface, and/or silvering/reflecting of the display surface at least in part or in portions, which allows to reduce the necessity of rotating the apparatus even with adverse light incidence on the display surface, and by specific representation of information directed with respect to a viewing angle of a user allows to increase data privacy and at the same time allows to properly read and represent the information even in adverse conditions.

In the case of representation of information depending on the viewing angle via controlled liquid crystals, for example at an orthogonal viewing angle with a tolerance of e.g. +/−10°, specific therapeutic parameters (e.g. kT/V, a HK value and the like) and setting options can be displayed, whereas, when viewing at an angle larger than e.g. 15° (20°, 25°, . . . ) up to e.g. 30° relative to the orthogonal line, general information such as duration of treatment, time and the like can be represented. Such representation can be configured both one-sided and two-sided and can be modified by the user via machine settings. In this case, the image representation may appear outside of the defined viewing angles as a mirrored or silvered or reflecting surface. This can be technically materialized, for example, by applying an electric voltage to liquid crystals which influence the direction of polarization of the light and, in this way, enable different displays and angles of insight.

As an alternative or in addition, polarization planes in combination with a prism structure (the latter for increasing polarization) can be adjusted to a fixed viewing angle so that scattered light is deflected into different directions in an improved manner. In this way, dazzling of the user may be reduced. In order to increase the degree of polarization, plural individual polarizations can be sequentially positioned.

Improved deflection of scattered light and reduction of dazzling of the user can further be obtained by antireflection coating with a coating of the display surface, for example in the form of an antireflecting coating (AR coating), with prisms, lenses, a rough surface and the like.

The degree of reflection of the AR coated surface may be achieved and, respectively, influenced by a destructive interference of the reflected rays. In favor of a reduction of reflection in this respect, for example, a non-absorbing layer (anti-reflection layer) operating according to the principle of destructive interference of a substance or a compound of substances having a predetermined refractive index and thickness can be evaporated onto a substrate. When said evaporated layer having a predetermined refractive index is adjacent to air, wave trains of the light reflected by the layer and the surface located there beneath can destructively interfere, if a phase condition and an amplitude condition are fulfilled as interference conditions. If the afore-mentioned interference conditions can be exactly fulfilled by one single layer having a uniform refractive index for one wavelength only, plural layers having different refractive indices may be applied as an altogether broad-band anti-reflection layer. It is further possible to suppress Fresnel reflections by laminating e.g. an LCD or LED panel directly, i.e. without any air gap, to a display surface, and/or to achieve antireflection coating by plasma etching.

Thus, antireflection coating is capable of improving transmission and contrast and of allowing good visibility of the screen or display content from plural, in the ideal case from all directions. A user can directly make changes of parameters even in the case of adverse light incidence before and during a treatment without initially having to rotate the monitor out of a viewing angle with glare or, respectively, glare angle. Specific silvering, polarization and/or change of contrast help to achieve the fact that confidential contents about a patient, for example, can be read from a particular position only. Visitors or other patients in this case are no longer able to easily view said confidential data, which allows providing increased and improved data privacy. Moreover, this configuration enables, e.g. in an interconnected dialysis center, parameter settings of a different machine to be displayed, as the latter can be read out by an authorized person (e.g. nurse) only. Thus, parameters of different patients can be easily compared without said information being visible to any third person.

Further advantages are achieved by an arrangement which alternatively or additionally provides a divisible monitor content. Divisible in the present context relates to the simultaneous representation of at least two different screen/display/monitor contents out of which only one of the screen/display/monitor contents is visible from a predetermined viewing angle or angular viewing range and/or from a predetermined distance or a predetermined angular distance range and only a different one of the screen/display/monitor contents is visible from a different predetermined viewing angle or angular viewing range and/or from a different predetermined distance or a predetermined angular distance range of the viewing direction. As an example, an alarm display or alarm message can be mentioned which is visible only at a quite large distance from the monitor or screen, whereas closely in front of the monitor a different display content which may contain menu navigation and detailed information, for instance, is visible or becomes visible when the monitor is approached. As afore-mentioned, in any case both display contents are represented so that for a single user varying his/her position the display content changes or for each of two users being located at different positions different information is readable from the same screen. For a first user who may be at a distance of several meters from the machine the afore-mentioned alarm message or alarm display is visible, and for a second user who may be positioned e.g. directly in front of the machine detailed information and a menu navigation are visible.

In detail, the afore-stated advantages are realized and an object is achieved by an apparatus for extracorporeal blood treatment comprising a body and a monitor unit which is fixed and cannot be orientated or at least not tilted relative to the body, hence it is adapted to be non-orientable or at least to be non-tiltable relative to the body, and includes a display unit, wherein the monitor unit includes a display content representation influencing device which is arranged to influence the visibility of information represented on the display unit. The display content representation influencing device in so far may be an upstream layer, disk or film for coating or silvering the representation, and/or a partial device or component of the display unit varying the visibility of the display content depending on the angle and/or the direction.

Advantages are resulting especially for a dialysis machine which may require an increased observation effort and increased demands to data privacy and in which displayed parameters and/or values are not intended to be freely accessible.

Of preference, the display content representation influencing device includes an antireflection coating means arranged on a display surface of the display unit which is arranged to coat at least a portion of the display surface. The coating means may be a reflection-reducing means that is arranged to reduce reflections. Advantageously, even in the case of adverse incidence of light on the display surface the necessity of rotating the apparatus may thus be reduced, and at the same time the information can be properly read and, respectively, represented even in adverse conditions. Thus, anti-reflection coating may improve transmission and contrast and may allow for good visibility of the screen or display content from plural, in the ideal case from all directions.

Of preference, the coating means is a film or coating disposed in the at least one portion on or in front of the display surface, or a film, a disk, a contrast changing means and/or a LED array disposed in front of the display surface of the display unit.

Of preference, the display content representation influencing device contains a silvering means/reflection means disposed on a display surface of the display unit which is arranged to specifically reflect at least a portion of the display surface. Advantageously, data privacy can be increased by representation of information which is targeted and, respectively, orientated with respect to a viewing angle of a user.

Of preference, the display content representation influencing device is a polarizing means or contrast changing means integrated in the display unit which is arranged to render represented display content readable from a predetermined position or viewing angle of a user only.

Of preference, the display unit is arranged to simultaneously display at least two different display contents and the display content representation influencing device is arranged to influence the visibility of the at least two display contents simultaneously represented on the display unit so that from a first viewing direction and/or distance of a first user to/from the display unit and from a second viewing direction and/or distance of a second user to/from the display unit a different one of the at least two display contents is visible.

Of preference, the display unit is configured as a dual-view display unit. A dual-view configuration advantageously enables different image contents to be presented for several persons who are looking at the display unit from different perspectives, directions or viewing angles.

Of preference, the display unit is configured as a split-screen display unit. The split screen configuration advantageously enables, with appropriate division of the area of the display unit, different image contents to be separately represented, for example also those which are made available via a network from a remote source, for example a doctor's workstation, from a service provider for maintenance or assistance purposes or from any other medical apparatus. In combination with the dual-view configuration it is further advantageously possible to keep such image contents invisible to unauthorized persons.

Of preference, the display content representation changing device is a filter adapted to be electronically controlled for changing its light transmission properties which is configured as a parallax barrier. Advantageously, in this way any viewing areas can be assisted and/or optimized for one or more viewers. For example, switchable and thus light-directing liquid crystals operating according to the principle of parallax barriers can be advantageously used, which liquid crystals are integrated in the structure of a display unit as a second liquid crystal layer ahead of or behind a panel and which can be directly controlled independently of an imaging display unit (TFT or LED panel). An angular difference between image axes may amount to 40°, for example, wherein in this case viewers can see different images from a distance of e.g. 50 cm from the left side and the right side.

### BRIEF DESCRIPTION OF THE DRAWINGS

The invention is best understood from the following detailed description when read in connection with the accompanying drawings. Included in the drawings are the following figures:

FIG. 1 shows, in a simplified top view, a medical apparatus comprising a display unit forming part of a monitor unit and including a viewing angle control in which different display contents are visible from different directions, according to a first embodiment;

FIG. 2 shows, in a simplified top view, a medical apparatus comprising a display unit forming part of a monitor unit and including a viewing angle control in which different display contents are visible from different distances, according to a second embodiment; and

FIG. 3 shows a cut-out schematic diagram of the display unit including a viewing angle control and optionally a coating and/or silvering layer application according to the first and second embodiments.

### DETAILED DESCRIPTION OF THE PREFERRED EMBODIMENTS

In the following description of figures, in the individual figures like or equally acting elements and/or components are denoted equally and/or with like reference numerals and appropriately are not redundantly described. In cases in which a subsequent embodiment functionally corresponds to at least one preceding embodiment, i.e. corresponding functions, arrangements and/or process or operating sequences are equally comprised, only differences shall appropriately be discussed.

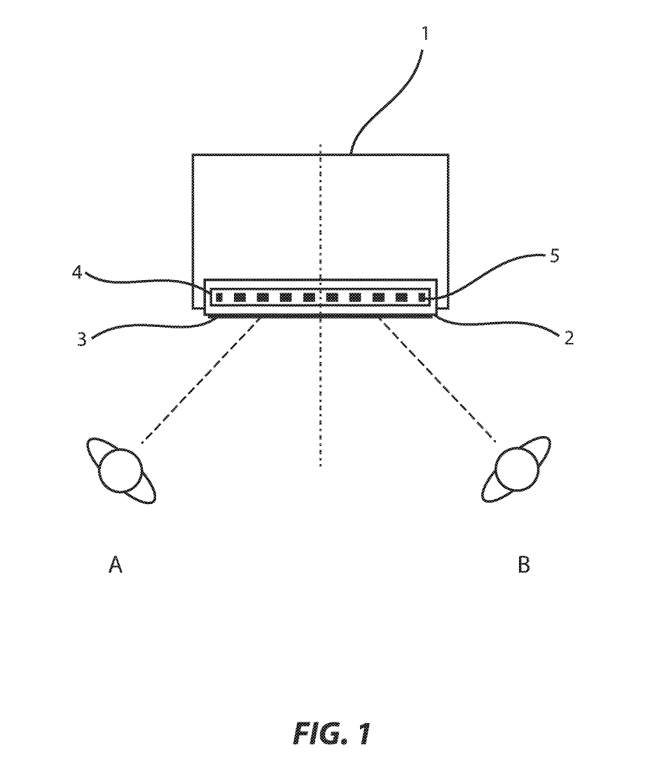

FIG. 1 illustrates, in a simplified top view, a medical apparatus 1 comprising a monitor unit 2 in which different display contents are visible from different directions according to a first embodiment. Hereinafter, the first embodiment is described by way of two different display contents and corresponding to two different directions A, B. However, there is no restriction hereto.

In the medical apparatus 1, especially in the present embodiment and in particular in a dialysis machine, a display unit or display device of the monitor unit 2 is configured or designed according to a so-called split-screen and/or dual-view principle and operates accordingly.

It is noted that, in the case of the dialysis machine, on the housing and, respectively, the body a number of components for carrying out a blood treatment are arranged to be functionally interacting in a manner known per se, such as control electronics, at least parts of the hydraulic system, pumps, heating means and, where needed, tanks or bags for selected operating substances, all of which are sufficiently known from the state of the art and therefore shall not be illustrated or described in detail.

The monitor unit 2 includes a display unit 4 in which viewing angle controllers 5 to be further specified are integrated and makes use of an individual (e.g. liquid crystal, TFT or LED) display screen (panel) for simultaneously representing at least two different still and/or moving images which are visible in an isolated manner from respective different viewing directions and/or distances (viewing perspective). In other words, from different perspectives a completely different image is shown to a viewer on the same screen. The viewing angle controllers 5 in this embodiment are means changing a light direction, for example an electronically controllable light direction changing filter or liquid crystals controllable as to their orientation by applying an electric voltage, and configure a display content representation influencing device.

In this embodiment, on the surface of the display unit 4 an at least partially reflection-reducing or antireflection-coating and/or silvering coat or layer 3 for the surface may be applied. Alternatively and/or simultaneously, a separate reflection-reducing or coating and/or silvering component may be provided in the form of a plate-shaped or disk-shaped element, for example, in front of the surface.

A reflection-reducing layer may be evaporated onto a substrate, for example as a non-absorbing layer functioning according to the destructive interference principle (anti-reflection layer or AR layer) of a substance or a compound of substances having a predetermined refractive index and thickness. When said evaporated layer having a predetermined refractive index is adjacent to air, wave trains of the light reflected by the layer and the surface located there beneath are destructively interfering, if a phase condition and an amplitude condition are fulfilled as interference conditions. If the afore-mentioned interference conditions can be exactly fulfilled by one single layer having a uniform refractive index for one wavelength only, plural layers having different refractive indices may be applied as an altogether broad-band anti-reflection layer.

It is further possible to suppress Fresnel reflections by laminating e.g. an LCD or LED panel directly, i.e. without any air gap, to a display surface and/or to obtain antireflection coating by plasma etching.

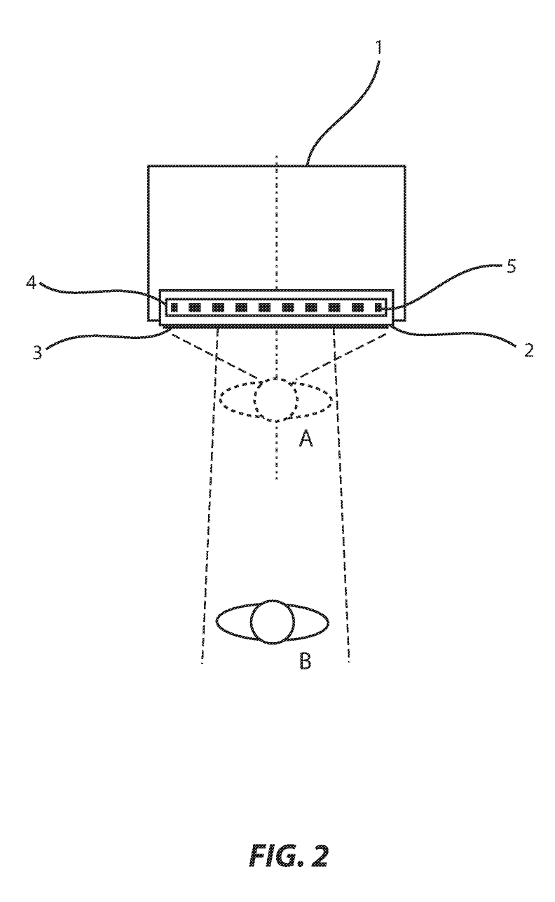

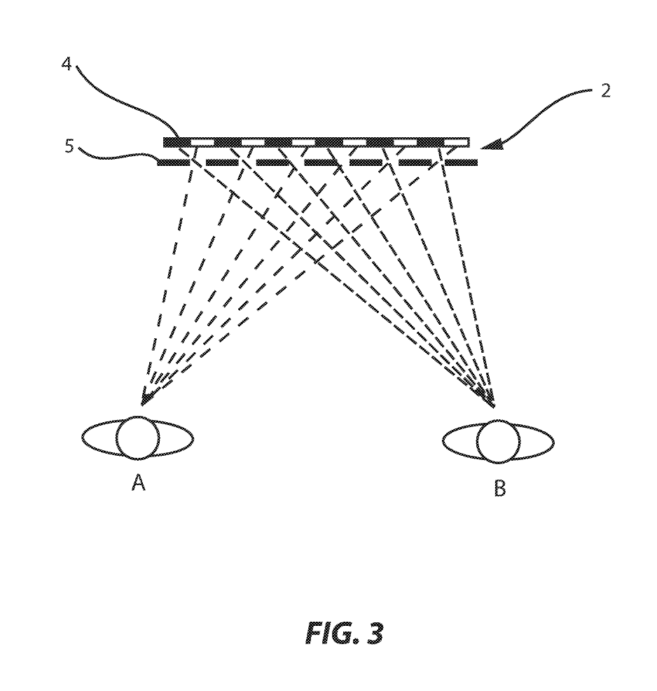

FIG. 2 illustrates, in a simplified top view, a medical apparatus comprising a monitor unit 2 in which different display contents are visible from different distances according to a second embodiment. The basic arrangement and functioning of the monitor unit 2 according to FIG. 2 corresponds to that of FIG. 1. An exemplary functioning of the monitor unit 2 according to FIGS. 1 and 2 shall be described hereinafter with reference to FIG. 3.

FIG. 3 illustrates a cut-out schematic diagram of a display unit 4 forming part of the monitor unit 2 including an angular view control and an optionally antireflection-coating and/or silvering layer application and in so far represents the principle of a parallax barrier according to which a screen or monitor represents two different images, to be sure, as indicated in the Figure by a sequence of rectangles alternately filled in black and white on a line (first image, rectangles filled in black, and second image, rectangles filled in white), but that, depending on the position of a person A or B, the latter will see only one of said images.

The display unit 4 including a viewing angle control described herein may be configured also in this embodiment according to a dual-view principle and/or a split-screen principle which permits to create two different representations of image contents for two viewers viewing from different directions.

For example, for this purpose so-called dual-stacked liquid crystal displays or LCD can be used in which any viewing areas can be supported and/or can be optimized for one or more viewers, and, with switchable liquid crystals functioning according to the parallax barrier principle which are integrated as a liquid crystal layer in the structure of a display unit (panel) and can be directly controlled independently of an imaging display unit (e.g. an LCD, TFT or LED panel), molecule strands of the liquid crystals are orientated by electric signals to form an aperture grille which controls the light (background light or backlight) to the left and to the right so that the light can be specifically directed to different directions.

In other words, in the embodiments described here at least two images can be simultaneously represented, wherein the light of individual pixels is directed, with the specific LCD layer being arranged in front of or behind the actual LCD, TFT or LED panel and functioning as filter, to the different directions, for example to a first direction A and to a second direction B ahead of the screen and a different image falls into the eyes of each of the persons (viewer/user) positioned in the first direction A or in the second direction B (see FIG. 3).

Therefore, on the display unit 4 the two images appear in different thin strips alternately for a left image and a right image. By reason of this aperture grille, pixels for the left image are visible from the left only, whereas to the right the grille only permits the view to the image parts of the right image. In this way, it is possible to represent two completely different contents on the same screen depending on the viewing angle or the direction. In this case, viewers looking from different viewing angles, i.e. perspectives (see FIG. 1, FIG. 3) or distances (see FIG. 2), at the monitor unit 2 see respective different image contents.

An angular difference between the two image axes may amount to 40°, for example, wherein in this case viewers can see different images from the left side and from the right side from a distance of 50 cm, for example. However, there is no limitation to said exemplary values.

If the same visual content is supplied to both sides, the display unit may also be used as a normal display unit.

As afore-mentioned, a change-over of the vision angle or viewing angle of the layer of switchable liquid crystals integrated in the structure of the TFT panel can be performed by electronically controlling the strands of the switchable liquid crystal molecules (by electric voltage) and thereby orientating the same in a predetermined manner so as to control the optical path of the background light. The double refraction of the liquid crystal material can be varied in this way so that light can emerge to the front only and, when viewing from the top or from the side, the field of view remains dark. If the voltage decreases again, the molecule strands again adopt an optically isotropic condition and let the light of the background lighting pass into any direction.

As afore-described, suitable switch cycles and suitable change-over and/or synchronization help to achieve the fact that a more distant user B can see a first display content visible at a small viewing angle only, whereas a user A standing in a predetermined manner in front of the display unit can see a second display content visible at a large viewing angle (wide angle). A possibly occurring overlap of the first and second display contents can be optimized or, respectively, suppressed with suitable equal pulsing and synchronization of the control of the display unit of the monitor unit 2.

Preferably, the medical apparatus 1 and, respectively, the dialysis machine may moreover include a detection device, such as a camera, an approximate sensor, a weight sensor, a person or face identifier, a card reader or a keyboard for entering an identifying sequence or the like (not shown) by which an approach of a user A or B can be detected. The wide-angle representation of the display unit then can be preferably activated only upon detection of the presence of the user A in front of the apparatus, wherein in this case information represented on the display unit can be visible from the distance only (and thus not readable in detail) without a user being positioned in sufficient vicinity, while reading close insights remain suppressed.

As afore-described, a medical apparatus comprises a housing and, respectively, a body, in the case of a dialysis machine for receiving operational components for the extracorporeal blood treatment, and a monitor unit including a display unit, wherein the monitor unit includes a display content representation influencing device which is arranged to influence the visibility of information represented on the display unit.

As advantages of the apparatus according to aspects of the invention, inter alia greater availability of information, the representation of different contents on a monitor, an increase in data privacy by limited viewing areas, the option of viewing, from each apparatus at a place where plural apparatuses are gathered and/or interconnected, parameter settings from a different apparatus, and the improved readability of monitor information even in adverse viewing and light conditions can be mentioned.

It is understood that the invention is not restricted to the described embodiments, but that, within the scope of protection defined by the following claims, nevertheless obvious combinations of at least parts of said embodiments, modifications and equivalents may result to those skilled in the art.

1-9. (canceled)

10. An apparatus for extracorporeal blood treatment comprising:
a housing for accommodating operational components for the extracorporeal blood treatment;
a body coupled to the housing; and
a monitor unit including a display unit and a display content representation influencing device, the monitor unit adapted to be non-tiltable relative to the body, wherein the display content representation influencing device is arranged to influence the visibility of information presented on the display unit.

11. The apparatus for extracorporeal blood treatment according to claim 10, wherein the display content representation influencing device comprises an antireflection coating disposed on at least a portion of a display surface of the display unit.

12. The apparatus for extracorporeal blood treatment according to claim 11, wherein the antireflection coating is a film or coating disposed on the at least a portion of the display surface, integrated into the display surface, or at least one of a film, a disk, a contrast changing means or a LED array arranged in front of the display surface of the display unit.

13. The apparatus for extracorporeal blood treatment according to claim 10, wherein the display content representation influencing device comprises a silvering means disposed on a display surface of the display unit which is arranged to silver at least a portion of the display surface.

14. The apparatus for extracorporeal blood treatment to claim 13, wherein the display content representation influencing device is a light direction changing filter, polarizing means or contrast changing means integrated in the display unit in front of or behind an imaging panel which is arranged to render represented display content readable only from a predetermined position or viewing direction of a user.

15. The apparatus for extracorporeal blood treatment according to claim 10, wherein
the display unit is arranged to simultaneously display at least two different display contents; and
the display content representation influencing device is arranged to influence the visibility of the at least two display contents simultaneously represented on the display unit so that from at least one of a first viewing direction or a first viewing distance of a first user to the first display unit only one of the at least two display contents is visible and from at least one of a second viewing direction or a second distance of a second user to the display unit only another one of the at least two display contents is visible.

16. The apparatus for extracorporeal blood treatment according to claim 10, wherein the display unit is configured as a dual-view display unit.

17. The apparatus for extracorporeal blood treatment according to claim 10, wherein the display unit is configured as a split-screen display unit.

18. The apparatus for extracorporeal blood treatment according to claim 10, wherein the display content representation changing device is a filter adapted to be electronically controlled for changing its light transmission properties.

19. The apparatus for extracorporeal blood treatment according to claim 18, wherein the filter is a configures as a parallax barrier.

20. The apparatus for extracorporeal blood treatment according to claim 10, wherein the monitor unit is adapted to be non-orientable relative to the body.

21. The apparatus for extracorporeal blood treatment according to claim 10, wherein the extracorporeal blood treatment apparatus is a dialysis machine.
